(19) United States
(12) Patent Application Publication
(10) Pub. No.: US 2019/0224349 A1
(43) Pub. Date: Jul. 25, 2019

(54) PREPARATION OF CARRIER CONTAINING RADIOLABELED COLLOID

(71) Applicant: Institute of Nuclear Energy Research, Atomic Energy Council, Executive Yuan, R.O.C., Taoyuan (TW)

(72) Inventors: Ying-Hsia Shih, Taoyuan (TW); Tsai-Yueh Luo, Taoyuan (TW); Cheng-Liang Peng, Taoyuan (TW)

(21) Appl. No.: 16/011,734

(22) Filed: Jun. 19, 2018

(30) Foreign Application Priority Data
Jan. 24, 2018 (TW) 107102503

(51) Int. Cl. A61K 51/12

(52) U.S. Cl. CPC A61K 51/1217

(57) ABSTRACT

The present invention relates to a preparation method of a carrier containing a radiolabeled colloid, which comprises the following steps: adsorption: putting a carrier into a radioisotope water solution, and reacting for 5-20 minutes to obtain a first solution; coating: adding a solvent capable of enabling a colloid to be formed into the first solution, and reacting for 5-20 minutes to obtain a second solution; and purification: centrifuging the second solution to form a first supernatant and a first precipitate, removing the first supernatant, cleaning the first precipitate with deionized water, centrifuging to form a second supernatant and a second precipitate, and finally, removing the second supernatant, wherein the residual second precipitate is the carrier containing the radiolabeled colloid.

### CROSS REFERENCE TO RELATED APPLICATION

This application also claims priority to Taiwan Patent Application No. 107102503 filed in the Taiwan Patent Office on Jan. 24, 2018, the entire content of which is incorporated herein by reference.

### TECHNICAL FIELD

The present invention relates to a preparation method of a carrier containing a radiolabeled colloid, and in particular to a technique for coating a radiolabeled colloid on nano-particles and micro-particles.

### BACKGROUND

Nano-particles and micro-particles have been applied to the drug delivery system for many years. There are some drug-carrying/carrier products in the market which have been applied to clinical use at present, such as DC bead Hepasphere embolization microspheres, Embozene micro-particles and yttrium-90 microspheres. DC beads are as follows: sulfo-modified PVA are made into hydrogel microspheres, and the hydrogel microspheres carrying doxorubicin are applied to transcatheter arterial embolization (TAE) for liver cancer; and Embozene micro-particles comprise a core composed of hydrogel and an external shell prepared from Polyzene, belong to microspheres with a larger core, and are used for tumor embolism with mass blood vessels and arteriovenous malformation (AVM).

Yttrium-90 microspheres (SIR-Spheres®microspheres) are of selective in-vivo radiotherapy, are resin microspheres with radioactive yttrium-90, and are prepared by adsorbing a water solution containing radioisotope yttrium-90 by a micro particle structure. Some research shows that the yttrium-90 microspheres can be used for treating liver metastasis tumors of colorectal cancer.

However, the yttrium-90 microspheres have the following problems: 1, the yttrium-90 microspheres are a material which cannot be degraded by microbes and cannot be absorbed and metabolized by the human body, and have the unspecific risk of staying in the body for a long time; and 2, the yttrium-90 microspheres are a product imported from Australia and cost is very expensive, so a single patient needs to spend about 0.6-0.8 million NTD for one course of treatment, and thus, only few patients can receive such expensive treatment.

In view of this, the present invention provides a preparation method of a carrier containing a radiolabeled colloid, which can solve the problems above.

### SUMMARY

The present invention aims to provide a preparation method of a carrier containing a radiolabeled colloid, and particular to a technique for coating a radiolabeled colloid on nano-particles and micro-particles.

To achieve the goals, the present invention discloses a preparation method of a carrier containing a radiolabeled colloid, which comprises the following steps: adsorption: putting a carrier into a radioisotope water solution, and reacting for 5-20 minutes to obtain a first solution; coating: adding a solvent capable of enabling a colloid to be formed into the first solution, and reacting for 5-20 minutes to obtain a second solution; and purification: centrifuging the second solution to form a first supernatant and a first precipitate, removing the first supernatant, cleaning the first precipitate with deionized water in an injection mode, centrifuging to form a second supernatant and a second precipitate, and finally, removing the second supernatant, wherein the residual second precipitate is the carrier containing the radiolabeled colloid.

The present invention also aims to provide a carrier containing a radiolabeled colloid, which is prepared by the preparation method of the carrier containing a radiolabeled colloid.

The efficacies of the present invention are mainly reflected in the following: 1, the preparation method of the carrier containing a radiolabeled colloid can effectively fix a radioisotope into the carrier, thereby enhancing the curative effects on the focus, for example, enhancing the slow-release effect of the drug; and 2, the preparation process is simple, and the cost is low.

### BRIEF DESCRIPTION OF DRAWINGS

FIG. 1 is a flow chart of the preparation method of the carrier containing a radiolabeled colloid in the present invention;

FIG. 2 is a microanalysis chart of Re-188 colloid micro-particles formed from different concentrations of stannous chloride (SnCl₂) in the present invention;

FIG. 3 is a drug dissolution release curve chart of the radioisotope Re-188 in the present invention;

FIG. 4 is a distribution diagram of the Re-188 radiolabeled colloid micro-particles in mice in the present invention;

FIG. 5a is a curve diagram of the volumes of tumors of the treated mice in the control group and experimental group in the present invention;

FIG. 5b is a curve diagram of the weights of the treated mice in the control group and experimental group in the present invention;

FIG. 5c is an external view of the volumes of tumors of the treated mice in the control group and experimental group in the present invention; and

FIG. 5d is a curve diagram of the survival rates of the mice in the control group and experimental group in the present invention.

### DETAILED DESCRIPTION OF DISCLOSED EMBODIMENTS

The present invention is only preferred implementations or embodiments of technical means to solve the problems, and is not to limit the implementation scope of the present invention. Equivalent variations and modifications made in accordance with the teachings of the claims of the present invention or according to the claims of the present invention are all encompassed by the claims of the present invention.

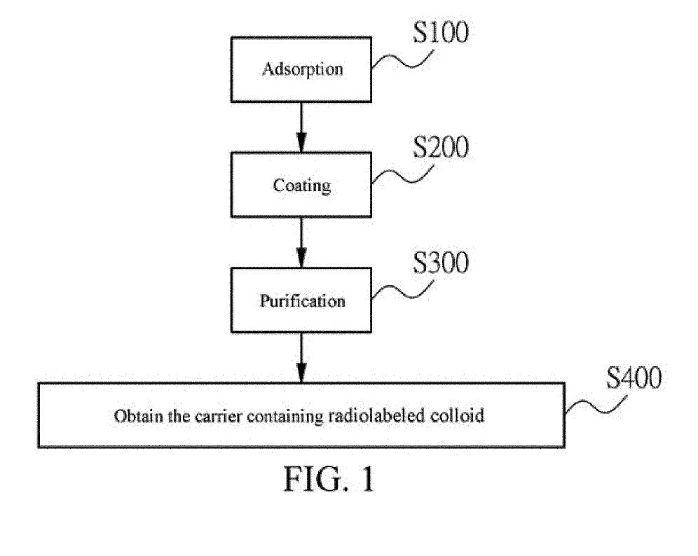

Please refer to FIG. 1. FIG. 1 is a flow chart of the preparation method of the carrier containing a radiolabeled colloid in the present invention. The preparation method comprises the following steps: adsorption (S100): putting a carrier into a radioisotope-containing water solution, and reacting for 5-20 minutes to obtain a radioisotope-carrier-containing water solution which is called a first solution, wherein the carrier is a nano-particle carrier or micro-particle carrier, the nano-particle carrier or micro particle carrier belongs to a water-soluble porous structure carrier, and the ratio of the carrier to the radioisotope-containing water solution is (1 to 1) to (1 to 100); coating (S200): adding a 15-35 mg/mL solvent capable of enabling the radioisotope water solution to form a colloid into the first solution, and reacting for 5-20 minutes to obtain a radioisotope-colloid-containing carrier solution with not high purity which is called a second solution, wherein the solvent capable of enabling the radioisotope water solution to form a colloid is a stannous chloride solution (SnCl₂); and purification (S300): centrifuging the second solution to form a supernatant and a precipitate, removing the supernatant, slowly cleaning the residual precipitate with deionized water in an injection mode, centrifuging to form a supernatant and a precipitate, and removing the supernatant, wherein the residual precipitate is the carrier containing the radiolabeled colloid (S400).

As mentioned above, the radioisotope water solution is a Re-188 radioisotope water solution. The above-mentioned centrifuging conditions are as follows: 2000 rpm, 10 minutes.

In addition, the steps of adsorption and coating are operated under the conditions of 300 rpm and 37° C.

To sum up, the preparation method of the carrier containing a radiolabeled colloid is a technique for carrying nano-particles and micro-particles into a radiolabeled colloid (radiocolloid), which is implemented by combining water-soluble porous-structure nano-particles and micro-particles with a radioisotope water solution and adding a chemical solvent capable of enabling the radioisotope water solution to form a colloid, wherein the chemical solvent can enable the radioisotope water solution to stay and be fixed in the pores of the carrier for some time and enable the formed colloid to fully fill the pore structure of the carrier. The carrier containing a radiolabeled colloid provided by the present invention is suitable for diagnosing or treating many clinical diseases, such as cancer, heart diseases, bone diseases, ophthalmic diseases, arthritis, etc.

### EMBODIMENTS

The following is to illustrate the test groups used in the embodiments of the present invention: Re-188 micro-particles and a Re-188 colloid are used as control groups for the following tests, and the experimental group is the Re-188 colloid micro-particles provided by the present invention, wherein preparation methods of the Re-188 micro-particles and Re-188 colloid in the control groups and the Re-188 colloid micro-particles in the experimental group are as follows:

Control Group: Re-188 Micro-Particles

Mixing micro-particles (50 mg) and a Re-188 radioisotope water solution (50-1000 mCi/mL), reacting under the conditions of 300 rpm and 37° C. for 10 minutes, centrifuging at the speed of 2000 rpm for 10 minutes, removing the supernatant, cleaning by slowly injecting 1 mL of deionized water, centrifuging at the speed of 2000 rpm for 10 minutes, and removing the supernatant, wherein the micro-particles left on the lower layer are Re-188 micro-particles.

Control Group: Re-188 Colloid

Mixing the 50-1000 mCi/mL Re-188 radioisotope water solution and a 1 mL of 35 mg/mL stannous chloride (SnCl₂), reacting under the conditions of 300 rpm and 37° C. for 10 minutes, centrifuging at the speed of 2000 rpm for 10 minutes, removing the supernatant, cleaning by slowly injecting 1 mL of deionized water, centrifuging at the speed of 2000 rpm for 10 minutes, and removing the supernatant, wherein the residual lower layer is the Re-188 colloid.

Experimental Group: Re-188 Colloid Micro-Particles

Mixing micro-particles (50 mg) and a Re-188 radioisotope water solution (50-1000 mCi/ml), and reacting under the conditions of 300 rpm and 37° C. for 10 minutes, wherein this step is an action of adsorbing the Re-188 radioisotope; adding 1 mL of 35 mg/mL stannous chloride (SnCl₂), and reacting for 10 minutes, wherein this step is also operated at the speed of 300 rpm at the temperature of 37° C.; after the reaction is finished, centrifuging for 10 minutes, removing the supernatant, cleaning by slowly injecting 1 mL of deionized water, centrifuging at the speed of 2000 rpm for 10 minutes, and removing the supernatant, wherein the micro-particles left in the lower layer are Re-188 colloid micro-particles.

### Embodiment 1: Re-188 Absorptivity of Re-188 Colloid Micro-Particles

Please refer to Table 1. Table 1 shows the Re-188 absorptivity of the Re-188 colloid micro-particles formed from different concentrations of stannous chloride, which indicates that a higher Re-188 carrying percent can be obtained after different sizes of micro-particles (74 μm, 104 μm) are added into 35 mg/mL stannous chloride (SnCl₂) to react.

TABLE 1
Re-188 absorptivity of Re-188 colloid micro-particles formed
from different concentrations of stannous chloride

| | Stannous chloride (SnCl₂) | | | |
|---|---|---|---|---|
| | Unit (mg/mL) | | | |
| | 0 | 15 | 25 | 35 |
| Re-188 colloid micro-particles | 19.37% | 56.43% | 66.55% | 74.88% |
| (74 μm) | | | | |
| Re-188 colloid micro-particles | 16.89% | 70.12% | 81.57% | 84.84% |
| (104 μm) | | | | |
| Re-188 colloid | — | 95.34% | 93.91% | 92.21% |

### Embodiment 2: Microanalysis of Re-188 Colloid Micro-Particles

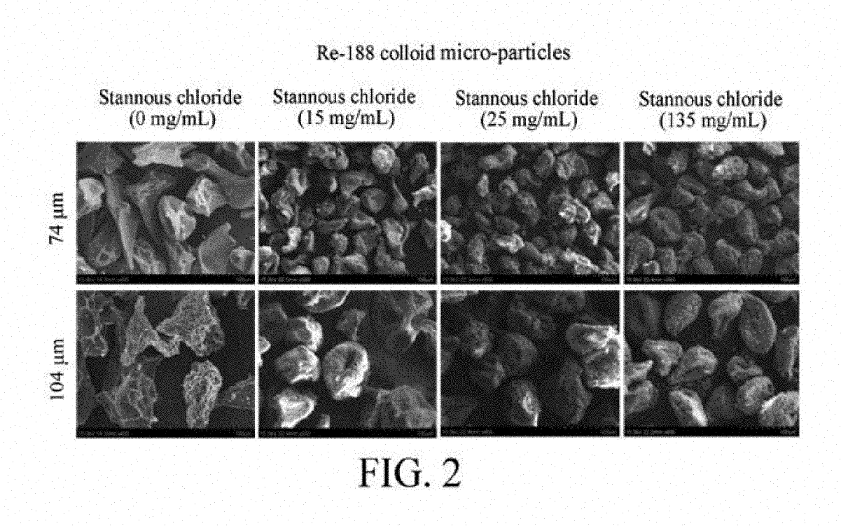

Please refer to FIG. 2. FIG. 2 is a microanalysis chart of Re-188 colloid micro-particles formed from different concentrations of stannous chloride (SnCl₂) in the present invention. It is detected that the porous structure of the micro-particles (74 μm, 104 μm) can be clearly seen before the stannous chloride (SnCl₂) is added, and the porous structure of the micro-particles (74 μm, 104 μm) is fully filled and coated after the stannous chloride (SnCl₂) is added, which indicates that the Re-188 colloid is carried into the porous structure of the micro-particles.

### Embodiment 3: Drug Dissolution Release Test of Radioisotope Re-188

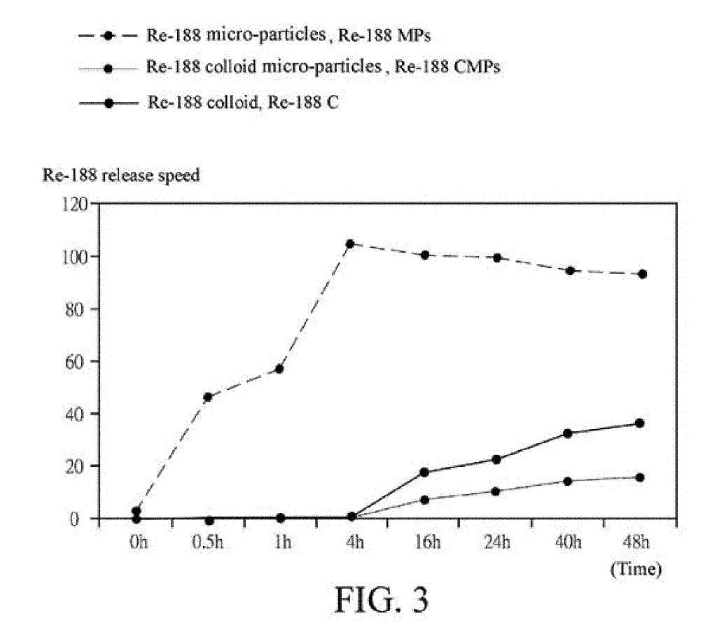

Please refer to FIG. 3. FIG. 3 is a drug dissolution release curve chart of the radioisotope Re-188 in the present invention. The results show that the Re-188 colloid micro-particles have the lowest release speed, the Re-188 colloid has the second lowest release speed, and the Re-188 micro-particles have the highest release speed.

### Embodiment 4: Distribution of Re-188 Colloid Micro-Particles in Mice

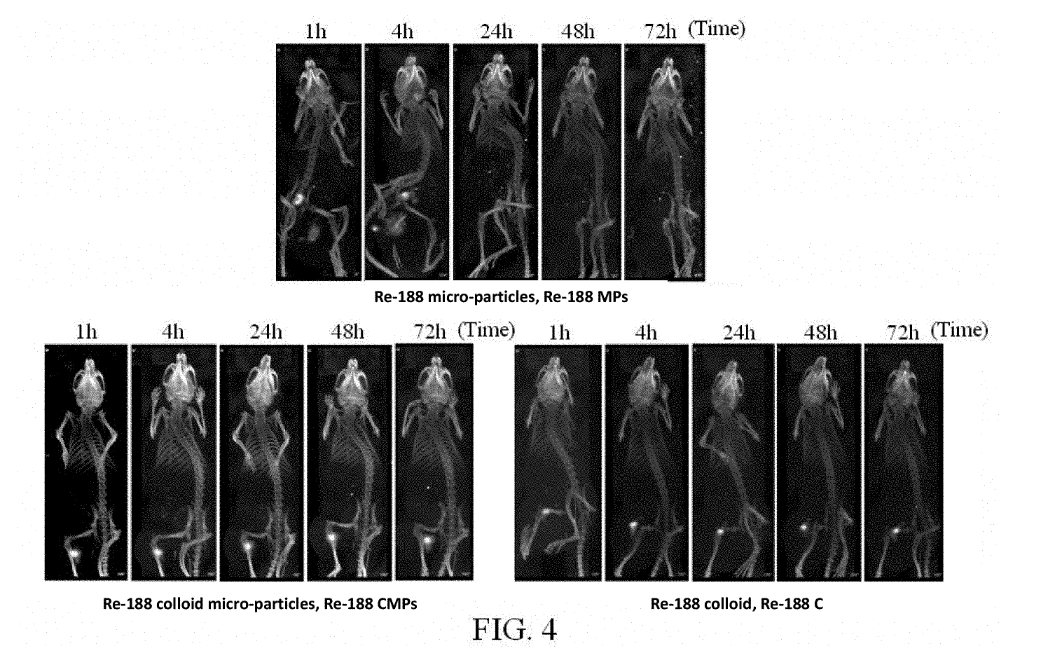

Please refer to FIG. 4. FIG. 4 is a distribution diagram of the Re-188 colloid micro-particles in mice in the present invention. Generally speaking, Re-188 micro-particles generated by simply adsorbing a Re-188 water solution by micro-particles can be easily excreted by the metabolic system of the human body. The test result shows that compared with the Re-188 micro-particles, the Re-188 colloid micro-particles and the Re-188 colloid can stay on the tumor site of the mouse for a longer time and cannot be easily metabolized and eliminated, which indicates that the Re-188 colloid micro-particles and Re-188 colloid can enhance the curative effect of radioisotopes on eliminating cancer cells on the tumor sites.

### Embodiment 5: Evaluation Test of Curative Effect of Re-188 Colloid Micro-Particles on Tumors

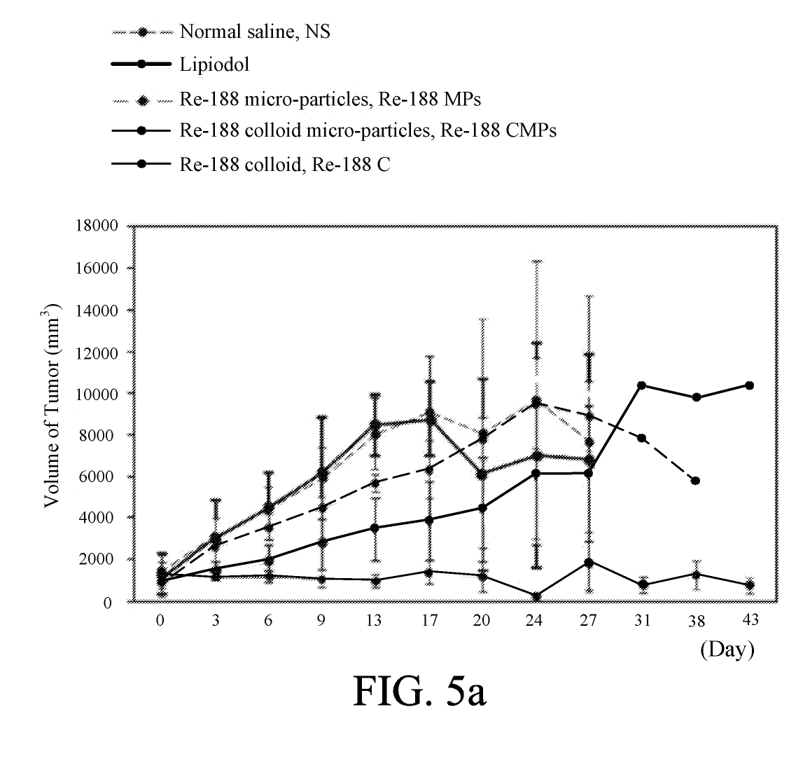

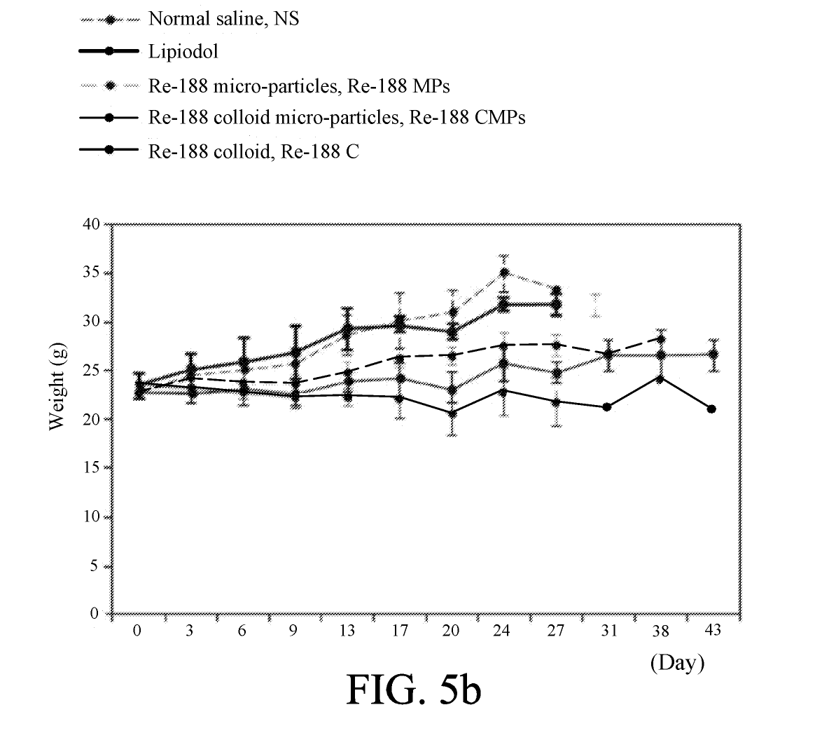

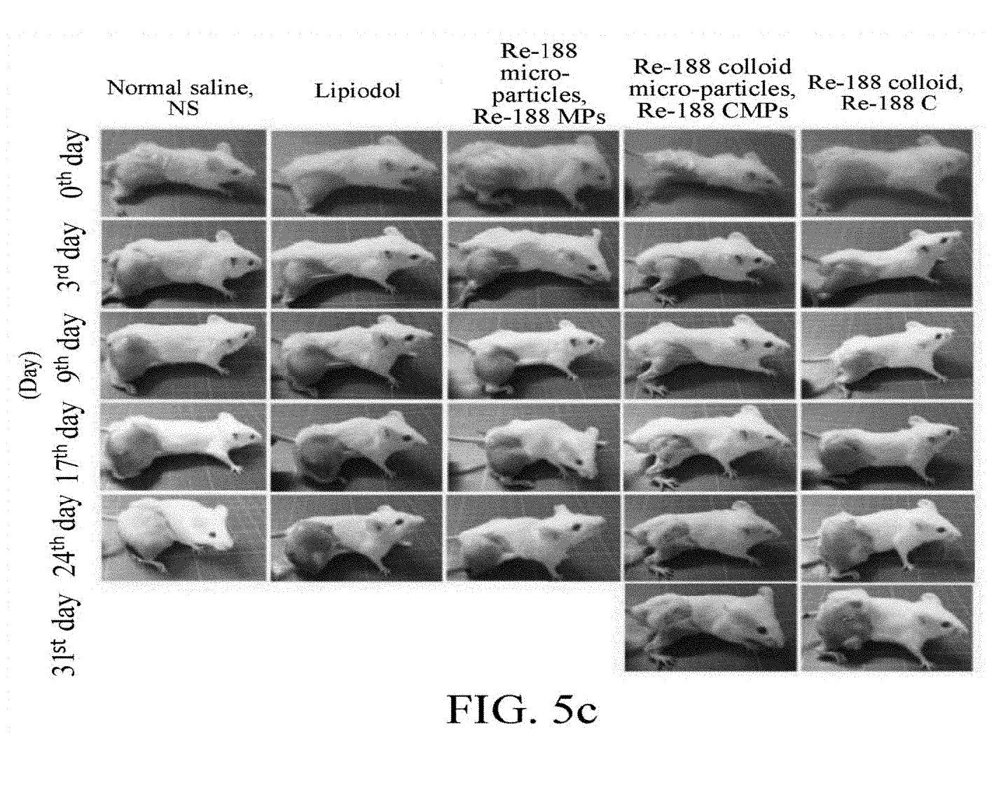

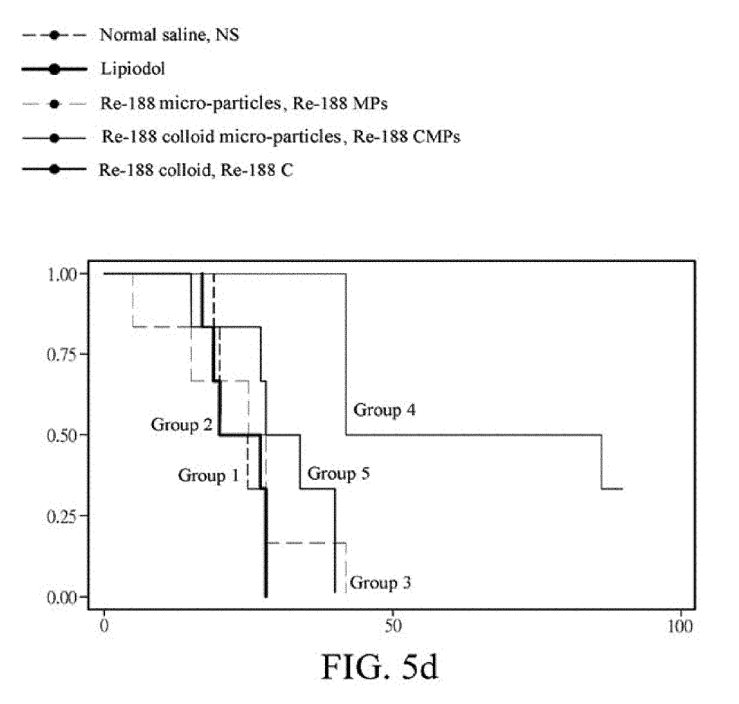

Please refer to FIG. 5a to FIG. 5d. FIG. 5a is a curve diagram of the volumes of tumors of the treated mice in the control group and experimental group in the present invention; FIG. 5b is a curve diagram of the weights of the treated mice in the control group and experimental group in the present invention; FIG. 5c is an external view of the volumes of tumors of the treated mice in the control group and experimental group in the present invention; and FIG. 5d is a curve diagram of the survival rates of the mice in the control group and experimental group in the present invention. The test result shows that the Re-188 colloid micro-particles have the best tumor inhibition and survival time prolonging effects, wherein saline (Group 1) and lipiodol (Group 2) are control groups, and Re-188 micro-particles (Group 3), Re-188 colloid micro-particles (Group 4) and Re-188 colloid (Group 5) are experimental groups

What is claimed is:

1. A preparation method of a carrier containing a radiolabeled colloid, comprising the following steps:
adsorption: putting a carrier into a radioisotope water solution, and reacting for 5-20 minutes to obtain a first solution;
coating: adding a solvent capable of enabling a colloid to be formed into the first solution, and reacting for 5-20 minutes to obtain a second solution; and
purification: centrifuging the second solution to form a first supernatant and a first precipitate, removing the first supernatant, cleaning the first precipitate with deionized water in an injection mode, centrifuging to form a second supernatant and a second precipitate, and finally, removing the second supernatant, wherein the residual second precipitate is the carrier containing the radiolabeled colloid.

2. The preparation method of the carrier containing a radiolabeled colloid of claim 1, wherein the carrier is nano-particles or micro-particles.

3. The preparation method of the carrier containing a radiolabeled colloid of claim 2, wherein the nano-particles and the micro-particles are of a water-soluble porous structure.

4. The preparation method of the carrier containing a radiolabeled colloid of claim 1, wherein the radioisotope water solution is a Re-188 radioisotope water solution.

5. The preparation method of the carrier containing a radiolabeled colloid of claim 1, wherein the solvent capable of enabling a colloid to be formed is a stannous chloride solution.

6. The preparation method of the carrier containing a radiolabeled colloid of claim 5, wherein the concentration of the stannous chloride solution is 15-35 mg/mL.

7. The preparation method of the carrier containing a radiolabeled colloid of claim 1, wherein the centrifuging conditions of the method are as follows: 2000 rpm, 10 minutes.

8. The preparation method of the carrier containing a radiolabeled colloid of claim 1, wherein the ratio of the carrier to the radioisotope water solution is (1 to 1) to (1 to 100).

9. The preparation method of the carrier containing a radiolabeled colloid of claim 1, wherein the steps of adsorption and coating in the method are operated under the conditions of 300 rpm and 37° C.

10. A carrier containing a radiolabeled colloid, prepared from the preparation method of the carrier containing a radiolabeled colloid of claim 1.
